(19) United States
(12) Patent Application Publication
(10) Pub. No.: US 2014/0088939 A1
(43) Pub. Date: Mar. 27, 2014

(54) EVALUATION OF ANIMAL PRODUCTS BASED ON CUSTOMIZED MODELS

(71) Applicant: AGRIDIGIT INC, Quebec (CA)

(72) Inventor: Dany Garant, Quebec (CA)

(73) Assignee: AGRIDIGIT INC, Quebec, QC (CA)

(21) Appl. No.: 14/115,320

(22) PCT Filed: May 2, 2012
(86) PCT No.: PCT/CA2012/050285

§ 371 Date: Nov. 1, 2013

Related U.S. Application Data
(60) Provisional application No. 61/481,280, filed on May 2, 2011.

(51) Int. Cl. G01N 33/12

(52) U.S. Cl. CPC G01N 33/12
USPC 703/2

(57) ABSTRACT

There is described a computer-implemented method for evaluating an animal product. The method comprises receiving measurement data of the animal product and selecting a generic mathematical model built from a plurality of corresponding animal products from a sample population of a given species or sub-species representing a variability of at least one aspect of a whole population. The generic mathematical model is parameterized by deformation using the measurement data of the animal product to generate a customized mathematical model of the animal product, and the animal product is evaluated based on the customized mathematical model.

### CROSS-REFERENCE TO RELATED APPLICATIONS

The present application claims priority under 35 USC 119(e) of U.S. Provisional Patent Application bearing Ser. No. 61/481,280 filed on May 2, 2011, the contents of which are hereby incorporated by reference.

### TECHNICAL FIELD

The present invention relates to the field of data management and evaluation systems and more particularly to the field of securely collecting, storing, and providing information concerning animal products. The information provided may be related to any aspect of evaluating an animal product on the basis of previously collected information for a similar species or sub-species and/or on the basis of presently collected information for a given animal product. This information may be useful and valuable for breeding, farm, feedlot and processing plant management, meat industry stakeholders, commercial transactions and for animal product evaluation.

### BACKGROUND OF THE ART

The process of getting a piece of meat from the farmer to the consumer is quite complex and involves many participants. The various steps involved are often disjointed, causing relevant information to be lost from one participant to the other.

Some examples of possible participants in the chain are breeding companies, farmers, butchers, consumers, meat packers, meat processors, retailers, governments, and even veterinarians. These participants all have different roles in the process, but they all use at least part of a same set of raw materials and information therefrom.

The quality of the information obtained by these different participants often depends on the equipment they use to obtain the information, i.e. those that can afford expensive equipment will obtain more precise information than those who cannot afford the expensive equipment. In addition, the differences in equipment being used to obtain the information causes a lack of consistency in the industry amongst the different participants.

For the meat industry to progress, there is a need to establish value-based payment and marketing systems, which rewards the producer for the quality and the real value of its work and its product, and orients breeding, feedlot and processing plant management based on consumer-driven market needs. As a result, value-based marketing systems based on the real value of the product are increasingly common in the industry. For these marketing systems to be effective, this requires a better integration, better exchange of information between various stakeholders so as to produce an animal that best meets the market requirements and also maximizes revenues for the different stakeholders of the value chain. This requires better tools and benchmarking to evaluate the characteristics of animals and animal products.

To reach higher integration in the food production chain, there must be higher trust between production chain members and this may be achieved by better animal products value estimation, which is an important component of the animal product industry. The market value of animals and animal products, however, is still generally based on the weight of salable lean meat, with very little regard for product quality in terms of nutritional composition or organoleptic attributes. Genetics and breeding of animals determine the quality and value of animals and the detailed composition of their carcasses affect taste and losses in the manufacture of specific products from these carcasses.

In addition, unlike traditional manufacturing, the meat industry has a reversed production process, thus creating different products from a single raw material, i.e. animals and ultimately their carcass. The challenge is to make better use of this raw material which shows significant natural variations because an animal is an organic product. The industry is dealing the best it can with this variability in terms of nutritional quality, weight, dimensions, shape, fat layer thickness, amount of meat etc., by sorting into groups, animals or animal products with similar characteristics. This reduction in variability by sorting could be even greater with the ability to obtain even more precise cost-effective quality metrics and attributes.

Therefore is therefore a need for an information sharing solution that can reach all of the different participants of the processing chain, while addressing each of their own needs in an adequate manner, and for a process that will improve use of raw materials throughout the chain.

### SUMMARY

There is described herein a method for objective measurement and sharing of the composition and quality information of animal products.

In accordance with a first broad aspect, there is provided a computer-implemented method for evaluating an animal product. The method comprises receiving measurement data of the animal product and selecting a generic mathematical model built from a plurality of corresponding animal products from a sample population of a given species or sub-species representing a variability of at least one aspect of a whole population. The generic mathematical model is parameterized by deformation using the measurement data of the animal product to generate a customized mathematical model of the animal product, and the animal product is evaluated based on the customized mathematical model.

Evaluating the animal product may include an evaluation on the basis of quality, grade, cut, composition constituents, spatial information, cooking, breeding, feeding, logistics, and other factors relating to the assessment of an animal product and/or the assessment of an animal (including an embryo or an egg) that will eventually become an animal product.

In accordance with another broad aspect, there is provided a data collection and evaluation system for animal products. The system comprises a memory comprising a plurality of generic mathematical models built from a plurality of corresponding animal products from a sample population of a species or sub-species representing a variability of at least one aspect of a whole population. An interface is adapted to receive measurement data of the animal product. The system also comprises a processor having an application coupled thereto which when run, causes the processor to select one of the generic mathematical models for the animal product; parameterize the generic mathematical model by deformation using the measurement data to generate a customized mathematical model of the animal product; and evaluate the animal product based on the customized mathematical model.

In accordance with yet another broad aspect, there is provided a computer-implemented method for creating a generic mathematical model for animal product evaluation. The method comprises receiving three-dimensional data representative of an outer surface and internal structures of a plurality of animal products from a sample population of a species or sub-species. Composition constituents of each one of the plurality of corresponding animal products are defined. A statistical variability analysis of at least one aspect of the composition constituents is performed and a three-dimensional model is reconstructed from a set of statistically analysed composition constituents.

In this specification, the expression “Grading data” is intended to refer to a set of descriptive terms describing measurement data features of the animal product that are useful to those involved in the production, fabrication and trading of animal products for pricing purposes, depending on established payment grids.

The expression “species” should be understood to mean a kind, variety, or type of animal without any limit to taxonomic classifications, thereby including a genus or a sub-genus. A “sub-species” is a further grouping within a species. Sorting is a grouping based on measurement and/or grading data.

The expression “animal product” is intended to refer to any animal or part thereof, including but not limited to the carcass, the skin, the organs, the viscera, the meat cut, the bones, the muscles, the tissues, and the fat. The animal product may be live or not, and may be beef, pork, chicken, lamb, and others.

The expression “cutting instructions” is intended to refer to any information related to separating the animal product into smaller components, such as where to position the cutting tool to begin the cut, which part of the animal product will yield the highest quality cut, which part of the animal product should be removed, which cutting tool to use, which cutting technique to use, etc.

The expression “measurement data” is intended to refer to any parameter or attribute that may be acquired or known for an animal product and used to characterize the animal product, such as weight, size, dimensions, internal structures, external structures, spatial information of the internal/external structures, physical properties of internal/external structures and their composition constituents, meat/fat texture, meat/fat color, meat/fat shear force, meat/fat shear density, PH, proteins, lipids, blood type, pedigree and breed, growth parameters, feed, medical history, genetic markers, and other elements that may have an impact on the overall quality of the animal product. The measurement data may relate to physical and/or behavioral characteristics taken on either a live animal, an embryo, a carcass, a cut of meat, animal organs/viscera, or any part thereof.

The expression “internal structures” is intended to refer to any physical element found sub-surface, such as organs, bones, meat, fat, muscles, blood, and others.

The expression “external structures” is intended to refer to any visible surface elements, such as visible internal structures revealed by cutting an animal product, or the skin and other elements found on the skin, such as tattoos, hair, branding marks, bruises, injection sites, wounds, scars, and others.

The expression “composition constituents” is intended to refer to any element contributing to the makeup of an animal product, such as meat, muscles, bone, fat, tissue, proteins, lipids, blood, water, and other elements that may have an impact on the overall quality or nature of the animal product.

### BRIEF DESCRIPTION OF THE DRAWINGS

Further features and advantages of the present invention will become apparent from the following detailed description, taken in combination with the appended drawings, in which:

FIG. 1 is a schematic showing some of the possible data inputs and outputs for the data collecting and exchanging system;

FIG. 2 is a block diagram illustrating an exemplary embodiment of a network comprising the data collecting and exchanging system;

FIG. 3 is a block diagram detailing the data collecting and exchanging system, in accordance with one embodiment;

FIG. 4 is a flowchart of a method for building a generic model of a given species, in accordance with one embodiment;

FIG. 5 is a flowchart detailing the step of entering image, measurement data and other parameters from the flowchart of FIG. 4, in accordance with one embodiment;

FIG. 6 is a flowchart of a method for obtaining information on a given animal using the generic model of a given species, in accordance with one embodiment;

It will be noted that throughout the appended drawings, like features are identified by like reference numerals.

### DETAILED DESCRIPTION

There is described herein a system and method for providing useful information to the different participants involved in taking a meat product from animal to consumer. The method and system allow farmers to receive feedback regarding their own livestock that may be used to improve the quality of the animal product produced from future livestock. The feedback information may be spatial or tissue-based, related to genetic factors, environmental factors, and other factors having an impact on producing healthy animal products that meet the demands of the market and the requirements of the consumers. Similarly, feedforward information may be provided to other participants in the production chain to obtain information regarding a given animal product in order to evaluate the animal product or utilize information therefrom at various levels with respect to specific parameters of the animal product. This information is obtained using a customized mathematical model for a given species or species. Customization is done by parameterizing a generic model built from a statistically representative sample population that represents the variability in a spatial domain of at least one parameter of a whole population. The mathematical model is deformed using measurement data obtained from the animal product under evaluation. Once customized, the model may be used to evaluate the animal product and share information with the meat industry stakeholders, as will be described in more detail below.

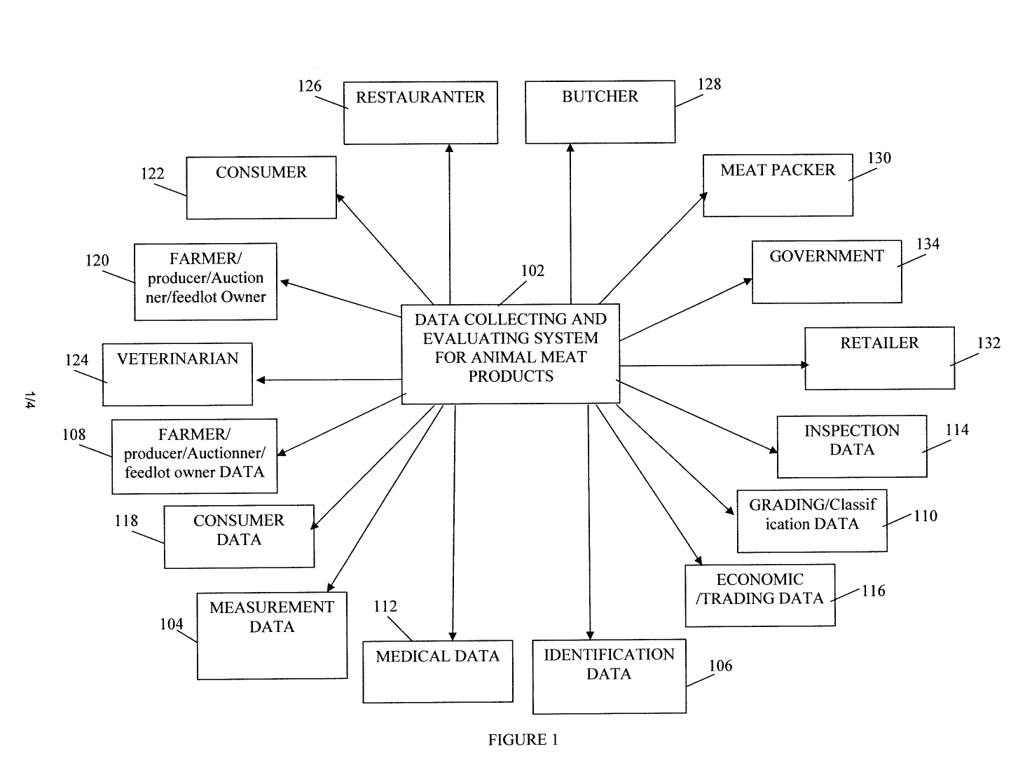

FIG. 1 is a schematic illustration of the various participants that can benefit from a system 102 for information collecting and sharing. The process of taking a meat product from animal to consumer usually starts with a farmer breeding the animal. The animals are fed and grown until they reach a desired maturity or they are sold to an intermediary party, such as feedlots and finishing farms, who specialize in bringing these animals to maturity. In some cases, farmers may trade or sell animals amongst themselves, for various economical reasons. Once the animal has reached maturity, the farmer or intermediary party will bring the animal to the slaughterhouse for slaughtering and evaluating. The slaughterhouse will purchase the animal from the farmer or intermediary party in accordance with the evaluation made. Some slaughterhouses having processing capabilities will perform the processing of the meat products themselves. In other cases, the slaughterhouse will sell the slaughtered animal to a meat product processor. Processed meat is then sold to butcher shops and/or supermarkets. Butcher shops and/or supermarkets will then offer the meat products to the consumer at a given price.

Meat production chain stakeholders data 108 relates to a wide variety of information, i.e. measurements, attributes, medical, identification, economic, grading, inspection and other data, gathered from the point of origin of the animal to the consumer. Examples include pedigree and breed, growth parameters, feed, medical history, genetic markers, and various attributes of the animal (geographic, biometric, etc).

Inputs to the system 102 may come from various sources. For example, measurement data 104 refers to physical and/or behavioral characteristics taken on either a live animal or embryo, a carcass, a cut of meat or animal organs/viscera. The measurement data may be obtained using internal or external measuring devices. The physical attributes, and other descriptive and health assessment information is generally termed in this application as the phenotypic information. Genetic information is termed in this application as the genotypic information. Generally, these are two distinct and differing sets of information. The phenotypic information may relate to observable physical or biochemical characteristics, as determined by both genetic makeup and environmental influences, while the genotype information may relate to the genetic makeup.

Surface measuring devices should be understood to refer to invasive and non-invasive devices that are used to obtain 1 D, 2D or 3D information of the visible envelope of at least part of an animal product. Examples of these devices are rulers, Video Image Analysis (VIA), PH meters, thermometers, color meters, genetic marker tests (DNA), sound/voice recorders and others. An example of 3D information is illustrated in U.S. Pat. No. 7,399,220, where the physical characteristics of livestock animals are measured using camera(s) strategically positioned to obtain data concerning volumetric, curvilinear (surface) and linear measurements. These devices may be contact or non-contact, and data acquisition may be automatic or manual. Sub-surface measuring devices should be understood to include invasive and non-invasive devices that provide information on the internal structures of the subject. Examples of these devices are opto-electronic probes, rulers used on exposed internal parts, Magnetic Resonance Imaging (MRI), Computed-Tomography (CT), X-Rays, Positron Emission Tomography (PET), Single Photon Emission Computed Tomography (SPECT), Bio Impedance devices, Ultrasound, Total Body Electrical Conductivity (ToBEC), Video Image Analysis (VIA) used on exposed internal parts, genetic marker tests (DNA), Magnetic Induction Spectroscopy (MIS) and others. These devices may be used manually, in an automated fashion, or in a semi-automated fashion and can acquire partial or complete internal information of a subject.

In some embodiments, the environment in which the surface and/or sub-surface measuring devices are used is one in which there may be a high level of humidity, high temperatures, and low or poor lighting conditions. The animal product from which data is being acquired may be moving and may require stoppage in the movement for data acquisition. Vibrations and minimal movement may be acceptable using certain measuring devices. Spatial orientation of the animal product may be favored using certain measuring devices. In some embodiments, the system is adapted to take into account the above conditions. For example, data acquisition may be performed without having to stop the animal products as they are displaced while suspended in the slaughterhouse. Acquisition time may be adapted to correspond to the speed at which the animal products are displaced. Additional lighting may be provided to take into account the poor lighting conditions.

Measurement data 104 may be provided by the farmer as part of the farmer data 108, or it may be provided separately by another participant, such as a butcher, a meat packer, or a retailer. Measurement data 104 may be associated, because of a priori knowledge or not, to a live animal, a carcass, a cut of meat, or viscera (organs), and may include biometric measurements, behavioral information, statistical deformable spatial configurations or composition mathematical models, tissue deformation models, and other complementary attributes information. The measurement data 104 may be used to estimate meat quality, quantity, position, dimension and shape.

When used with a payment grid, grading data 110 may allow determination of the amount owed to the farmer or any other meat production chain stakeholders for a purchase, for the purposes of value based marketing.

Identification data 106 may be used for traceability of the animals. This can include batch or individual identification and/or registration data. Some examples are implants, biopsy tags, visual markers, RFID tags, unique 3D features, and any other information that can be used to positively identify an animal, carcass, or animal product later on down the processing chain.

Medical data 112 may also be provided by the farmer as part of the farmer data 108, or it may be provided separately by another participant, such as a veterinarian or any caregivers. Medical data 112 refers to any physical, pathological, or other type of data relating to the health and well-being of the animal, as well as any past treatments administered to the animal.

Inspection data 114 refers to carcass/animal/animal products defects, viscera (organs) defects, hygiene information, and others. Carcass defects may be assessed using biometric measurements (such as magnetic induction spectroscopy or tomography) and visual information. Inspection data 114 may also include parasites, microbes, pathogens, odors, and fecal contamination (such as listeria, E. Coli, etc). Inspection data 114 may be acquired using tools such as fluorescence, reflectance/transmittance (UV-VIS-NIR), and Raman lasers, testing samples, and others. Pathogens and diseases may be identified using these tools and important data regarding the hygiene of the animal, carcass, cut of meat, or viscera (organs) is obtained. Viscera defects may also be identified using information such as biometric measurements, visual information, magnetic induction, and others. Identification of such defects can affect quantity, quality, and shape estimations for animal organs.

Economic data 116 refers to market price/demand information. Examples of such information includes stock exchange data, sales data, profitability data, and any other type of information that relates to enhancing or optimizing the financial value of the animal, carcass, or cut or meat, or viscera (organs).

Consumer data 118 refers to any attributes that may be noted by the consumer and that can be useful for future evaluation or estimation. For example cooking information, tenderness of a cut or meat, freshness of the meat, flavor, juiciness or any organoleptic information, etc.

Once the various data has been collected from the different sources, the system 102 is adapted to use the data in a plurality of ways. Accurate anatomical measurements for use in, for example, determining quality characteristics, veterinary inspection and/or processing of the animal product, may be obtained. The obtained anatomical measurements or quality characteristics may be used in subsequent processing of the animal product, e.g. for sorting and/or favorable cutting in predetermined parts that are optimally cut off in terms of e.g. price and customer wishes. Some possible uses for the data are breeding evaluations, feedlot evaluations, live animal and slaughter plant evaluations, and carcass evaluations. Reference is made to U.S. Pat. No. 7,399,220 for further details on these evaluations.

The surface of the animal product may be digitized and a set of points and zones of interest may be extracted using changes in colors, tints, texture, shape, and other physical properties. This imaging procedure also allows various defects of the animal product to be identified, such as tattoos, hair roots, bruises, skin problems, lung adhesions, injection sites, atrophied muscles, fat covers, wounds, etc.

By using inspection data and/or the deformable spatial configuration/composition model to determine parts of an animal product that have defects, it then becomes possible to estimate the quantity/part of the animal product that may have to be removed. The inspection data may allow to identify not only the animal product defects such as bruises, but also the point in time when it was caused or occurred; for example at the farm, during transportation or at the slaughterhouse. Alternatively, or in addition thereto, the model may be used to estimate the amount/part that has been removed or is missing and to estimate its characteristics (i.e. weight, volume, attributes, value, etc). By having a model that represents an animal product standard dressing presentation and deforming this model to the actual animal product, parts that are missing or should not be on the actual animal product may be identified by comparing the deformed generic model and the animal product volumetric representation. This becomes very valuable information when the time comes to grade the carcass, cut of meat, or viscera (organs) and evaluate its monetary value.

In another example, animal product profit optimization refers to a determination of how to enhance the profit obtained from a given animal product using information such as meat quality, quantity and shape estimations, meat cuts, trade value, client orders, or any type of historical or forecast data. The system 102 may therefore comprise an application capable of taking this data as input and upon applying an animal product profit optimization algorithm, advise a user of the best way to separate the animal product into different products in order to enhance his or her profits. An example of an existing system that may be used in combination with the system described herein is the Opti-profit™ system of NavAnalytics™.

In yet another example, the system 102 is adapted to perform animal product cutting automation optimization, which refers to guidance on how to best cut the animal product to maximize yield and minimize waste. Anatomy estimations and cutting equipment may be used to achieve this. The anatomy estimations may be generated using tissue deformation models, statistical 3D virtual models, and biometric measurements. More details on animal product cutting optimization may be obtained from U.S. Pat. No. 7,850,512, WO2008/010732, and WO2008/0200107.

Another application that can be provided in the system 102 is for the generation of optimized cooking instructions. The optimized way of cooking a given cut of meat may be customized using information such as meat quality, quantity and shape estimations, and complementary attributes information. It may be that beef from a given genotype should be cooked longer and slower due to the toughness of the specific meat cut, while the same cut of beef from another genotype should be cooked less long. In addition, the thickness, marbling grade, and cut of the meat may affect the optimized cooking instructions.

Other applications may include estimating animal posture, physical capabilities, and/or robustness. This information can then be used to predict weight support as a function of the dimensions of the skeletal frame, for example. Applications for estimating animal anatomy or capabilities at a later point in the animal's life or future condition of an animal product, or for the purposes of breeding two animals, are also possible. A chicken egg is a specific example of this, whereby the volume of the yolk or the fat content may be used to estimate characteristics of an eventual live chicken and/or its carcass. Predicting the anatomy of an offspring may be done by combining the parameterized model of the father and the mother. Augmented reality may be used for processing or cutting of the animal or for clinical interventions. It may be more cost-effective to have a butcher use vision enhancing technology or guiding instructions than to acquire robots/machines that are designed to mimic human skills. Automatic shape or anatomical landmark identification for traceability purpose, organ or viscera modeling, and sorting/fine branding are other examples of applications that may be integrated with the present system. Various other applications for optimizing and enhancing the different steps of the processing chain and addressing the needs of the different participants will be readily understood by those skilled in the art.

Referring back to FIG. 1, some of the different participants who may benefit or utilize the system 102 are presented. The farmer 120 may use the system 102 for example, for branding or certification purposes, payment, grading, growth strategy, feed strategy, attributes, and inspection or statistical purposes. A consumer 122 may use the system 102 for example, for obtaining cooking instructions or identifying attributes of a cut of meat. A veterinarian 124 may use the system 102 for example, for the purposes of evaluation of an animal that is sick, needs care or for clinical interventions. A restaurant 126 may use the system 102, for example, for cooking, branding, or pricing. A butcher 128 may use the system 102, for example, for cooking, branding, or pricing. A meat packer 130 may use the system 102, for example, for carcass value optimization, cutting automation or optimization, payment, growth strategy, feed strategy, attributes, and inspection. A retailer 132 may use the system, for example, for cooking, branding, attribute differentiation or pricing. A government official 134 may use the system 102, for example, for inspection, grading, or statistical purposes.

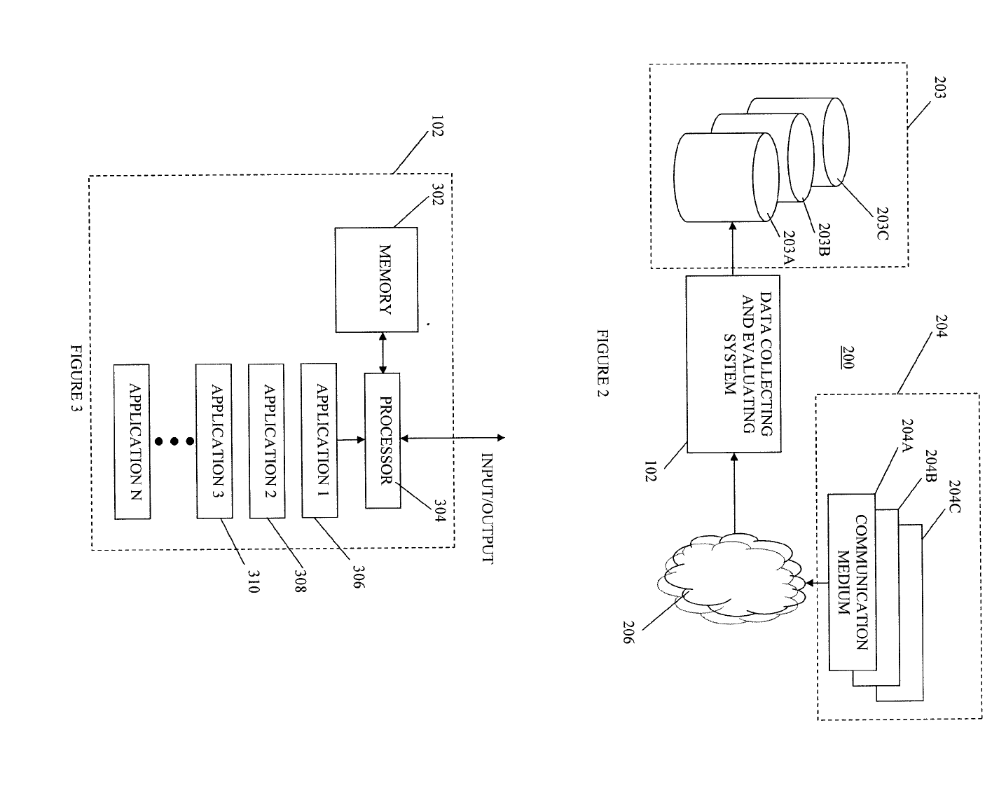

Referring to FIG. 2, there is illustrated a block diagram of an exemplary embodiment of a network 200 comprising a system for collecting and sharing information 102. One or more databases 203a, 203b, 203c (collectively referred to as 203) contain the various information referred to above regarding the collected data. More than one database 203a, 203b, 203c may be accessed by the system 102. In one embodiment, one database 203a may contain information related to only a single species of animal. In another embodiment, one database 203a may contain information related to all of the animals from a same farmer. The system for collecting and sharing information 102 is accessed by a communication medium 204a, 204b, 204c (collectively referred to as 204), such as a telephone, a computer, a personal digital assistant (PDA), an iPhone™, etc, via any type of network 206, such as the Internet, the Public Switch Telephone Network (PSTN), a cellular network, or others known to those skilled in the art. The system for collecting and sharing information 102 receives requests from the communication medium 204, and based on those requests, it accesses the database 203 to retrieve the requested information and provide it to the user via the communication medium 204. When a user transfers data in database 203 to another location or to another user, this may be done via the network 206, which can be the same network as that used to access the system for collecting and sharing information 102 or a different network.

FIG. 3 illustrates the system for collecting and sharing information 102 of FIG. 2 as a plurality of applications 306, 308, 310 running on a processor 304, the processor being coupled to a memory 302. It should be understood that while the applications presented herein are illustrated and described as separate entities, they may be combined or separated in a variety of ways.

The database 203 may be integrated directly into memory 302 or may be provided separately therefrom and remotely from the system for collecting and sharing information 102. In the case of a remote access to the database 203, access may occur via any type of network 206, as indicated above. The various databases described herein should be understood as collections of data or information organized for rapid search and retrieval by a computer. They are structured to facilitate storage, retrieval, modification, and deletion of data in conjunction with various data-processing operations. They consist of a file or sets of files that can be broken down into records, each of which consists of one or more fields. Users may retrieve database information through queries. Using keywords and sorting commands, users can rapidly search, rearrange, group, and select the field in many records to retrieve or create reports on particular aggregates of data according to various rules. The databases may be any organization of data on a data storage medium, such as one or more servers.

In one embodiment, the databases are secure web servers and Hypertext Transport Protocol Secure (HTTPS) capable of supported Transport Layer Security (TLS) is the protocol used for access to the data. Communications to and from the secure web servers may be secured using Secure Sockets Layer (SSL). An SSL session may be started by sending a request to the Web server with an HTTPS prefix in the URL, which causes port number 443 to be placed into the packets. Port 443 is the number assigned to the SSL application on the server. Identify verification of a user may be performed using usernames and passwords for all users. Various levels of access rights may be provided to multiple levels of users.

Alternatively, any known communication protocols that enable devices within a computer network to exchange information may be used. Examples of protocols are as follows: IP (Internet Protocol), UDP (User Datagram Protocol), TCP (Transmission Control Protocol), DHCP (Dynamic Host Configuration Protocol), HTTP (Hypertext Transfer Protocol), FTP (File Transfer Protocol), Telnet (Telnet Remote Protocol), SSH (Secure Shell Remote Protocol), POP3 (Post Office Protocol 3), SMTP (Simple Mail Transfer Protocol), IMAP (Internet Message Access Protocol), SOAP (Simple Object Access Protocol), PPP (Point-to-Point Protocol), RFB (Remote Frame buffer) Protocol.

The memory 302 accessible by the processor 304 receives and stores data, such as all the exemplary data described above and any other information used by the system for collecting and sharing information 102. The memory 302 may be a main memory, such as a high speed Random Access Memory (RAM), or an auxiliary storage unit, such as a hard disk, a floppy disk, or a magnetic tape drive. The memory may be any other type of memory, such as a Read-Only Memory (ROM), or optical storage media such as a videodisc and a compact disc.

The processor 304 may access the memory 302 to retrieve data. The processor 304 may be any device that can perform operations on data. Examples are a central processing unit (CPU), a front-end processor, a microprocessor, a graphics processing unit (GPU/VPU), a physics processing unit (PPU), a digital signal processor, and a network processor. The applications 306, 308, 310 are coupled to the processor 304 and configured to perform various tasks as explained below in more detail. An output may be transmitted to a communication medium 204.

The image data provided in the database may be represented under various forms. For example, in discrete form, exact 3D individuals or tables with limited measurements may be provided. In continuous form, various statistical methods may be used (such as principal component analysis, neural networks, interpolation/extrapolation from tables with limited measurements, and others) to represent the data. Other parameters regarding the representative population sample may also be provided to the system, as will be explained in more detail with regards to FIG. 5.

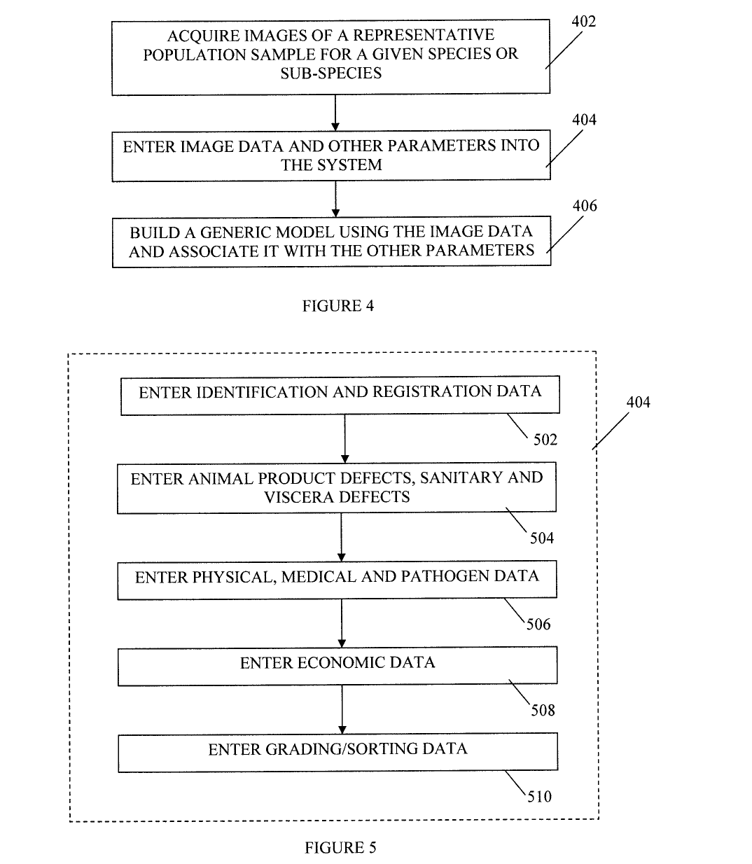

The steps performed by an exemplary application 306 running on the processor 304 is illustrated in the flowchart of FIG. 4. In this method, a generic deformable model of an animal product from a given species is built using a statistically representative population sample of the animal product from this species. In a first step 402, data of the representative population sample is acquired. The data may be three-dimensional data representative of an outer surface and of internal structures of the animal products. Various precise sub-surface imaging tools may be used for this acquisition step. For example, ultrasounds, PET, CT scans (Computed Tomography, X-ray laminography), and MRI are some non-invasive imaging techniques, while slicing and acquisition by photogrammetry and manual dissection and 3D acquisition are examples of invasive imaging techniques.

In one embodiment, the image data corresponds initially to 3D CT-scans and is converted into compact representations that contains information about body shape, such as a volumetric representation. The image data is entered into a database or another form of memory 404. In the case of digital images, these may be partitioned into multiple segments in order to simplify and/or change the representation of the image into something that is more meaningful and easier to analyze. Image segmentation may be used to locate objects and boundaries (lines, curves, etc.) in the images. Some exemplary segmentation techniques are contour detection, thresholding, clustering, compression-based, histogram-based, region-growing, partial differential equation-based, graph-partitioning, watershed, model-based, multi-scale, semi-automatic, neural networks, and atlas. Segmentation may be used to locate tumors and other pathologies, or to study the anatomical structure of the animal.

In some embodiments, an initial step of volumetric representation correction and alignment may be performed by first smoothing the surface using, for example, a Taubin filter to eliminate noise while minimizing distortion of the original geometry. To minimize misalignment, the models may be translated so that their centers of gravity are aligned. The volumetric representations of the body components are then re-oriented so that the coordinate system corresponds to the principal axes of their tensor of inertia.

In the case that a voxelisation technique is used, segmentation may first be used to separate the animal product surface into different body segments, whereby different criteria are applied to the different body segments when the consistent volumetric representations are generated. The corrected and aligned volumetric representations of at least part of the animal product 3D CT-scans are aligned inside a volume of fixed dimensions, which is sampled to a set of voxels. A 3D model is then characterized by an array of signed distances between the voxels and their nearest point on the body surface. Correspondence is achieved by comparing for each voxel the signed distances attributed to different models. Surfaces are then reconstructed from the volumetric description using, for example, the marching cubes algorithm. Volumetric representations allow correspondence between different models to be achieved with or without using anatomical landmarks or markers. This correspondence is needed in order to use the different models to generate a generic model, as will be explained in more detail below.

Alternatively, another way to create consistent volumetric representations is to build morphable bodies from the non-morphable 3D meshes. We perform feature-based parameterization using Radial Basis Functions (RBF), followed by surface-based parameterization using error minimization methods as will be detailed later in the generic model parameterization techniques. In that case, segmentation may also be used here to separate the animal product morphable consistent volumetric representation into different body segments, whereby separate PCA are applied to the different body segments to have richer expressiveness than conventional whole body PCA.

Valuable information may be extracted from the volumetric models by performing a Principal Components Analysis (PCA). To apply PCA to the volumetric models, a vector Ψ is formed for each model, where each element of the vector is the signed distance from a voxel to the surface of the model. The average over N models is given by

$$\overline{Ψ} = ( 1 / N ) {\sum }_{i = 1}^{N} {Ψ}_{i} .$$

The deviation vectors Φᵢ=Ψᵢ− Ψ̅ are arranged in a matrix A=[Φ₁Φ₂ . . . Φ_{N}]. The PCA of the matrix A generates a set of non-correlated eigenvectors uᵢ and their corresponding variances λᵢ. The eigenvectors are sorted according to the decreasing order of their variances. Each vector Φᵢ can be approximated as

$${{Φ}^{⋒}}_{i} \approx {\sum }_{j = 1}^{M} {c}_{ij} {u}_{j} ,$$

where 0≦M≦N and cᵢⱼ=Φᵢ·uⱼ. In other words, every model can be reconstructed by the linear combination of a subset of the eigenvectors. The quality of the reconstruction can be evaluated by the fraction Σᵢ₌₁^{m}λᵢ/Σᵢ₌₁ⁿλᵢ, representing the percentage of the variance spanned by the eigenvectors chosen for the reconstruction. Studying the variance of the models using PCA allows a form of 3D shape variation characterization, which may yield information on height, weight, fat content, bone mass, and other parameters affecting the quality, quantity and/or price of an animal product. In addition, specific parameters or attributes affecting the organoleptic quality of an animal product may be included in the generic deformable spatial configuration/composition model by associating local tissue properties or attributes with a statistical approach to the voxels within the model. That way, the model contains information on quality attributes distribution over the complete anatomy of at least part of an animal/animal product.

Referring back to FIG. 4, a generic statistical spatial configuration or composition 3D virtual model of the anatomy and the attributes of an animal population may be built 406 using the image data, brute or processed. Creation of the generic model may be performed using the following steps: 1—acquisition of external (visible) surface data, or acquisition of internal data and creation of surface data from internal images (e.g. CT-scans); 2+3—alignment of the 3D surfaces reconstructed from image data with a common coordinate system; computer graphical sectioning of the models if needed; 4—extraction of contours from the image sections, combining and averaging of extracted contours, and consistent 3D reconstruction of the averaged contours if using the voxelisation approach or feature-based and surface-based parameterization if using the landmarks approach; 5—and Principal Component Analysis. Other techniques for creating a statistical virtual model or virtual spatial representation of a set of imaged data will be readily understood by those skilled in the art.

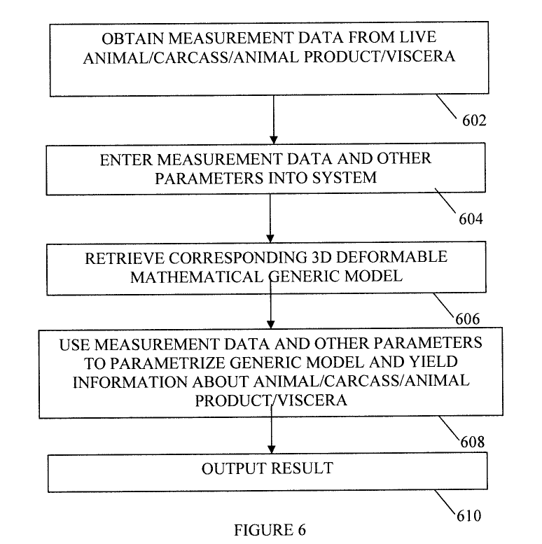

The virtual spatial representation allows a user to obtain specific measurements for the complete anatomy or body composition of all of the primary types of cuts or viscera, such as weight, fat content, dimensions, quality, and value. This will then allow a uniform grading system, as will be explained in more detail with regards to FIG. 6. As many statistical virtual models as there are animal populations of interest may be built and stored in the system 102. Models may be built and classified according to just species, or according to species and other distinguishing characteristics, such as geographical region, sub-species, genotype or phenotype category. By having multiple models in the system 102, it may be used with a wide variety of meat products, such as beef, chicken, pork, lamb, etc.

In one embodiment, the generic model is a morphable model built using a statistical approach and the measurement data corresponds initially to 3D scans obtained from the animal product. The 3D scan is then used as a set of geometric constraints in order to construct the best surface that satisfies these constraints.

In some cases, the 3D image data is incomplete or of low quality. Various repair techniques may be used to improve the quality of the data or compensate for missing data. For example, an initial step may be performed by first smoothing the surface using, for example, a Taubin filter to eliminate noise while minimizing distortion of the original geometry.

Improper alignment of the animal product during the scanning process may also lead to noise that affects the image data. To minimize misalignment, the models may be translated so that their centers of gravity are aligned. The 3D scans are then re-oriented so that the coordinate system corresponds to the principal axes of their tensor of inertia.

Segmentation may be used to separate the animal product surface into different body segments, whereby different criteria may be applied to the different body segments when the volumetric representations are generated in the case that a voxelization technique is used.

Hole-filling of the models may then be performed. The models may be sliced by intersecting horizontal planes with the edges of surface triangles. The hole-filling step then consists in connecting points that are connected to at most one other point so that each slice is converted to a set of closed curves. The voxelization technique based on a signed distance map, as previously explained in the generic model surface representation creation step, may then complete the repair process.

Another way to compensate for incomplete or low quality image data (e.g. 3D point clouds) is to deform a water-tight surface template to best match the current image data. That way the hole-filling process is eliminated and we get surfaces ready for further statistical analysis.

Alternatively, one can deform the complete generic spatial configuration or composition model itself, associated with a tissue deformation model, to best match the measurement data. That way, we already get the generic model ready for outputting results if no other parameterization is needed.

External surface parameterization of the surface template or the generic model itself using the animal product surface acquisition may be done using a two step approach: a rough deformation step using translation, rotation, and scaling, followed by a fine mapping step.

For example, a rough deformation step may be done using a Radial Basis Function (RBF) network. An RBF network can be stated as an interpolation: let pᵢεR³ and qᵢεR³, i=1 . . . n, be two sets of n landmarks (here n=85), which serves as the input. The source landmarks pᵢ lie on the generic model and target landmarks qᵢ correspond to features of the set of measurement data. Three RBF networks, one for each coordinate, are established to build the mapping:

qᵢ=f(pᵢ),i=1, . . . ,n (1)

Since this function is defined over the volume spanned by the landmarks, it can be used to deform all vertices on the body surface. This mapping can then be expressed by a radial basis function, i.e. a weighted linear combination of n basic functions defined by the two sets of landmarks:

$$f ( {p}_{i} ) = {\sum }_{j = 1}^{n} {w}_{j} {Φ}_{j} ( {p}_{i} ) \qquad ( 2 )$$

When computing the mapping coefficients, input points will be the landmarks and when doing the deformation, input points will be the vertices in the influence region of certain landmark from a 328K high-resolution generic model. This linear system is solved using a standard LU decomposition with pivoting. After training and computing the weight vector, new positions of those non-feature vertices are calculated by using this RBF network with their initial positions. The RBF transformed model maintains the same topology as the generic model.

In the mapping defined by Equation (1), there are several radial functions, which could be applied for model deformation. A first one is a thin-plate spline: Φⱼ(r)=r² log(r), where r is the Euclidian distance between the feature point and the input point.

A second possible radial basis function is a multi-quadrics function: Φⱼ(r)=(r²+s²)^{1/2} where s is called a stiffness constant, which controls the local or global effects of the landmarks. A third possible radial basis function is a Gaussian function: Φⱼ(r)=exp(−r²/c²), where c is the only parameter.

After RBF transformation, the generic model has been deformed closer to a target model in shape, pose, and height. When performing fine mapping, only data and smoothness errors are taken into account. Data errors may be defined as

$${E}_{d} = {\sum }_{i = 1}^{m} {dist}^{2} ( {T}_{i} {v}_{i} , Γ ) ,$$

where m is the number of non-marker vertices in U, and the dist( ) function computes the distance between a vertex on U and its closest vertex on Γ. Smoothness errors may be defined as

$${E}_{s} = {\sum }_{\{ i , j \vert \{ {v}_{i} , {v}_{j} \} ∈ edges ( U ) \}} {‖ {T}_{i} - {T}_{j} ‖}^{2} .$$

The smoothness error is not defined for the smooth surface but for the actual deformations to the generic model.

The diffusion process of transformations extends this deformation to neighbouring vertices. The result is that deformations for an area of nearby vertices are maintained as close as possible. The definition of a smoothness error minimizes the deformation over the template surface and thus prevents adjacent parts of the template surface from becoming aligned to disparate parts of the target surface. The overall error is defined as a weighted sum of these two errors: E=aE_{d}+bEₛ, where a and b are two weights for the data and smoothness errors. By setting two different weights for data and smoothness error, and by minimizing the overall error after running the optimization, we get the fine mapping results.

Other animal product surface parameterization techniques to get consistent surface representation may be used, such as Feynman parameterization, Schwinger parameterization, and solid modeling.

In one embodiment, the image acquisition means used to parameterize the generic model is electromagnetic induction tomography (EMIT) or spectroscopy (EMIS). By performing localized EMT, the conformation and/or internal attributes of a carcass can be evaluated. Other possible estimations are total lean meat, weight and yield of lean meat, intramuscular fat, water holding capacities, and carcass anatomy (muscles, fat, bones, etc).

Biometric and anthropometric measurements may also be made and used as parameterization parameters for the generic model. From this information, the animal species can be identified, and segmentation and virtual cutting of the generic spatial configuration/composition may be done to estimate the relative weight, muscle/fat content, and local tissue properties for example.

The following is a detailed exemplary method for developing a generic model as described herein. In a first step, a significant number of animal products representing the variability of the population are collected. This may be done in collaboration with specialists, such as butcher shops or slaughter houses. Some points of interest, such as anthropometric points or visible portions of the carcass may be noted. Sub-surface imaging tools, such as X-ray laminography, is used to scan the animal product. Measurement data associated with tissue components may be obtained using the same imaging tool or using other acquisition technologies in order to correlate all of the measurement data with the generic model to be created.

In one embodiment, the animal products are processed at different moments in time, i.e. at different stages of the growth process such as embryo, egg, live animal, meat cut, aged meat cut, etc. Measurement data is acquired from the animal product at the different stages of the growth process using various combinations of acquisition tools. The measurement data is then segmented into the different composition constituents, such as muscles, skin, bones, fat, tendons, etc using various image segmentation techniques.

Once segmented, a statistical model is chosen to represent the variability of the geometry of the animal product, such as PCA, MDS, LDA, LGE, etc. Another statistical model may be chosen to represent the variability of another aspect of the animal product, such as any of the measurement data related to non-geometric aspects. The chosen statistical models are then used to create the generic model by translating the measured data into useful values for the animal product.

FIG. 5 is a flowchart illustrating various types of parameters that can be entered into the system 102 in addition to the acquired image data. Identification and registration data of the animal may be provided 502. Animal product defects, sanitary information, and viscera defects may also be provided 504. A wide variety of physical, medical, and pathogen data associated with one or more of the animal products can be entered 506. Economic data, relating to market price, market demand, or Stock Exchange information may also be provided 510. The data entered may be post-slaughter or pre-slaughter. In the case of pre-slaughter, some possible information may relate to feeding, growth promoters, sex, age, genotype, phenotype and breed of the animal. With regards to post-slaughter data, some possible information may relate to meat temperature, fat layers, ossification, hanging methods, carcass washing, meat/fat texture, meat/fat color, PH, meat/fat shear force, consumer test panel information (organoleptic attributes), meat aging, genotype, phenotype, marbling, stress hormones quantity, cooking methods, and electrical stimulation. All of this information are used to create quality/quantity/condition attributes for the animal products.

In one embodiment, there is provided in the system 102 an application 308 for determining information with regards to a given animal product. FIG. 6 illustrates the steps for this method, whereby the generic model built in accordance with the method of FIG. 4 is used. The given animal product from which information is sought is of a same species as at least one of the generic models built and provided in the system 102. However, it should be noted that this is not a requirement. At the slaughterhouse, the butcher shop, the meat packer, or even the retailer, measurement data of the animal product may be obtained. This may be done using the same acquisition tool as that used to build the generic model, For example, if CT images were used to build the model, then CT images are acquired for the given animal product. Alternatively, measurement data may be obtained using a different tool. In the case of a different tool, there may be a correlation between the tool used for image acquisition of the sample population and the tool used to obtain measurement data of the carcass, animal products or viscera. The measurement data may correspond to data obtained via any type of data acquisition tool, or it may correspond to data obtained using manual tools such as a ruler, a scale, PH meter, EMIT, EMIS, etc.

The measurement data, for example 3D external surface data, total weight, etc, is entered into the system 102, as well as any other parameters or attributes of the animal product 604. The generic model that corresponds to the animal product is retrieved from the system 606. For example, a search may be done to find the model for beef raised in Austin, Tex., and a generic model meeting these search parameters may be retrieved. Using the measurement data acquired for the given animal product, the generic model is parameterized in order to yield the requested information about the given animal product 608.

Segmenting the animal product into individual parts (corresponding to body parts, types of cuts, etc) before performing the parameterization may result in more precision. In one embodiment, the entire morphable animal product model is pre-segmented by planes passing through landmarks in the generic spatial configuration/composition model. That way several individual parameterization and segmentation steps may be made a priori in order to yield results more quickly.

In one embodiment, the application 308 is for determining a grading of the animal product in order to associate a value thereto. In another embodiment, application 308 is for performing animal product cutting optimization. Using the generic model, specific parameters of the animal product and the measurement data for the given animal product are used to parameterize the generic model. The generic model is thereby forced to represent the real animal product being evaluated. For example, if the model is for pork animal products that weigh between 70 and 120 pounds and the given animal product weighs 100 pounds, then the model can be made more precise with this information. This precision will yield a more precise value for evaluating the animal product in terms of quality, quantity, or condition. For example, an animal product that weighs more than another with the same animal product volume may infer more meat and less fat deposit within the product.

In one embodiment, there is more than one model built for a given species, each model having been created using a different image acquisition tool. For example, one model is generated using ultrasound, another model is generated using CT, and another model is generated using PET. This provides added flexibility to the user of the system 102 in that they are not limited to only one imaging technique when no correlation is provided between the different image acquisition tools. Alternatively a model may be constructed using more than one image acquisition tool, such as CT and PET. Also alternatively, the different attribute acquisition tools are correlated and therefore, the tool used to acquire the image data from the sample population may be different from the tool used to obtain the measurement data.

Once the appropriate measurement data has been acquired, the corresponding generic model is retrieved 604. Using the measurement data and specific parameters of the given animal product 608, the model is parameterized and animal product cutting instructions or animal product grading data is provided for the given animal product 610.

In another embodiment, the application 308 may be used in a similar fashion to obtain optimized cooking instructions, to determine animal product profit optimization, to estimate the size of parts that need to be removed or have been removed on the animal product due to various defects, and for other known processes used in the animal meat product industry.

The present system allows the user to obtain more precise information without needing to resort to more expensive tools. The more information is gathered early on in the breeding of the animals, the more precise the information can be later on in the processing chain without the need for expensive tools or equipment. Precise information may be obtained for an animal product coming from an animal that was part of the representative population sample, or one that was not part of the representative population sample. In addition, precise information may be obtained for live animals as well, the live animals being independent or not from those used to build the generic model. A generic model may also be successfully parameterized to represent an animal product that is outside of the sample population variability range or that does not belong to the model database. In addition, a parameterized generic model may be added to the generic model database itself to continuously refine the database if needed. The generic model allows the user to isolate each muscle of the animal individually and obtain information therefrom.

The system 102 may be customized with the number of applications desired. For example, a complete version of the system 102 may include ten or more applications that can be used by different participants in the processing chain, while a partial version may include only a subset of the total number of applications available. Other examples of applications running on the system for animal products are as follows: classifying in accordance with universal meat standards, lean content determination, establishing marbling scores, assessing meat yields, extracting and analyzing grading features, determining salable yield and trimmable fat, determining tenderness, quantification of muscle, subcutaneous fat and intramuscular fat, etc. It will be understood that many known processes may be integrated into the system 102 such that the use of specific parameters for a given animal product will result in useful information when applied to a generic model that corresponds to the given animal product.

The system may be provided as a web-based system available to all parties using a network such as the Internet. The system may have various levels of access to information, with user profiles being configured by a system operator/manager. The ability to assess the quality of animal products using the present system not only provides opportunities for producers, but also for nutritionists to adapt animal nutrition to improve lean meat production, their efficiency and the quality characteristics of meat.

While illustrated in the block diagrams as groups of discrete components communicating with each other via distinct data signal connections, it will be understood by those skilled in the art that the embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present embodiment.

It should be noted that the present invention can be carried out as a method, can be embodied in a system, a computer readable medium or an electrical or electro-magnetic signal. The embodiments described above are intended to be exemplary only. The scope of the invention is therefore intended to be limited solely by the scope of the appended claims.

1. A computer-implemented method for evaluating an animal product, the method comprising:
receiving measurement data of the animal product;
selecting a generic mathematical model built from a plurality of corresponding animal products from a sample population of a given species or sub-species representing a variability of at least one aspect of a whole population;
parameterizing the generic mathematical model by deformation using the measurement data of the animal product to generate a customized mathematical model of the animal product; and
evaluating the animal product based on the customized mathematical model.

2. The computer-implemented method of claim 1, wherein evaluating the animal product comprises determining a quantity of at least one composition constituent of the animal product.

3. The computer-implemented method of claim 1 wherein evaluating the animal product comprises determining a quality of at least one composition constituent of the animal product.

4. The computer-implemented method of claim 1, wherein evaluating the animal product comprises determining at least one of a shape and a dimension of at least one composition constituent of the animal product.

5. The computer-implemented method of claim 1, wherein evaluating the animal product comprises determining a spatial position of at least one composition constituent of the animal product.

6. The computer-implemented method of claim 1, wherein evaluating the animal product comprises determining at least one of a part of the animal product that needs to be removed and a part of the animal product that has been removed.

7. The computer-implemented method of claim 1, wherein evaluating the animal product comprises determining optimized cutting instructions for the animal product.

8. The computer-implemented method of claim 1, wherein evaluating the animal product comprises determining optimized cooking instructions for the animal product.

9. The computer-implemented method of claim 1, wherein evaluating the animal product comprises determining at least one of optimized breeding, feeding, and logistical instructions for the animal product.

10. The computer-implemented method of claim 1, wherein evaluating the animal product comprises determining at least one feature of the animal product at a future moment in time.

11. The computer-implemented method of claim 1, wherein evaluating the animal product comprises determining at least one feature of the animal product at a past moment in time.

12. The computer-implemented method of claim 1, wherein evaluating the animal product comprises determining a unique identifier for traceability of the animal product.

13. The computer-implemented method of claim 1, wherein evaluating the animal product comprises determining a probability for at least one feature of the animal product.

14. The computer-implemented method of claim 1, wherein receiving measurement data comprises receiving at least one of weight and dimensions of the animal product.

15. The computer-implemented method of claim 1, wherein receiving measurement data comprises receiving at least one of economic, transactional, and commercial data for the animal product.

16. The computer-implemented method of claim 1, wherein receiving measurement data comprises receiving at least one of medical data and inspection data for the animal product.

17. The computer-implemented method of claim 1, further comprising associating a monetary value to the animal product as evaluated.

18. The computer-implemented method of claim 1, wherein selecting the generic mathematical model comprises selecting a mathematical model corresponding to a same species or sub-species as the animal product and based on data acquisition methods different from those used to acquire the measurement data of the animal product.

19. The computer-implemented method of claim 18, further comprising providing the generic mathematical model.

20. The computer-implemented method of claim 19, wherein providing the generic mathematical model comprises:
receiving three-dimensional data representative of an outer surface and internal structures of the plurality of corresponding animal products from the sample population of the species or sub-species;
defining composition constituents of each one of the plurality of corresponding animal products;
performing a statistical analysis of a variability of at least one aspect of the composition constituents; and
reconstructing a three-dimensional model from a set of statistically analysed composition constituents.

21. The computer-implemented method of claim 20, wherein defining composition constituents comprises differentiating fat, meat, bones, and skin.

22. The computer-implemented method of claim 20, wherein defining composition constituents comprises segmenting the three-dimensional data.

23. The computer-implemented method of claim 21, wherein defining fat, meat, bones, and skin comprises processing the three-dimensional data for a first one of the fat, meat, bones, and skin, removing pixels corresponding to the first one of the fat, meat, bones, and skin from the three-dimensional data, and proceeding sequentially with a second one of the fat, meat, bones, and skin, a third one of the fat, meat, bones, and skin, and a fourth one of the fat, meat, bones, and skin.

24. The computer-implemented method of claim 21, wherein defining skin comprises connecting segmented skin structures together.

25. The computer-implemented method of claim 20, further comprising displaying the three-dimensional model by color-coding the composition constituents for visual recognition.

26-31. (canceled)
